(19) United States
(12) Patent Application Publication
(10) Pub. No.: US 2002/0161333 A1
(43) Pub. Date: Oct. 31, 2002

(54) Catheter shut-off valve

(76) Inventor: Ronald B. Luther, Newport Beach, CA (US)

Correspondence Address: KNOBBE MARTENS OLSON & BEAR LLP, 2040 MAIN STREET, FOURTEENTH FLOOR, IRVINE, CA, 91614, US

(21) Appl. No.: 09/843,624

(22) Filed: Apr. 27, 2001

(51) Int. Cl. A61M 5/178; A61M 5/00

(52) U.S. Cl. 604/167.01; 604/167.02

(57) ABSTRACT

A device for preventing fluid flow out of a patient when a medical component is withdrawn from a catheter connected to the patient. The device includes a tubular hub and a clamp slidably mounted on the hub. A clamp arm is normally biased into a position in which it squeezes the cannula to prevent flow therethrough. The arm is either self biased or urged into closed position by a resilient element. Connecting the upstream end of the hub to the medical component produces relative movement between the hub and the clamp that causes cam surfaces on those members to bias the clamp arms into an open position that permits fluid flow through the cannula.

### BACKGROUND OF THE INVENTION

1. Field of the Invention

This invention relates to medical catheters in general, and specifically to a device and method for sealing a catheter from unwanted fluid flow.

2. Description of the Related Art

In the medical field it is often necessary to insert a catheter into a portion of the human body such as the bloodstream. One significant problem with implanting a catheter in the bloodstream of a patient is the tendency of blood to flow through the catheter upon removal of the insertion needle, stylet, trocar, or guidewire used in the procedure of piercing the body and placement of the catheter therein. Once the passageway between the bloodstream and the catheter opening is opened, blood tends to flow out of the body. Additionally, Luer connections such as intravenous feeding mechanisms occasionally become disconnected by the patient (intentionally or accidentally) thus allowing blood to flow from the patient out of the catheter. This unwanted blood flow out of the patient is obviously an undesirable result. It is therefore desirable to have a device which seals a catheter from fluid flow in one or both directions upon removal of the insertion needle, stylet, trocar, or guidewire.

Many solutions to the above-stated problem have been suggested. For example; U.S. Pat. No. 5,405,323 teaches a catheter check valve assembly which incorporates a duckbill valve and a manually operable separator. U.S. Pat. No. 4,449,693 discloses a valve of resilient tubing into which a stopper having an oval sealing ring is placed. U.S. Pat. No. 5,073,168 teaches a y-adapter with a check valve formed from conformable sheets. U.S. Pat. Nos. 5,112,301; 5,156,600 and 5,167,636 also teach other types of catheter check valves.

Some existing bi-directional catheter check valves rely on a differential fluid pressure across a membrane in order to seal the catheter from fluid flow. This will unfortunately not solve the problem of accidentally disconnected Luer connections, as blood pressure is typically higher than atmospheric air pressure, thus blood would leak out of such a valve in the absence of a second fluid. Others of the check valves described in the above-mentioned patents generally share the disadvantage that they require a conscious action to fully close the valve and seal the cannula from allowing blood to flow out. This extra step can be forgotten, thus leaving the catheter open to unwanted fluid flow.

It is desirable to have a check valve which will always be closed when the Luer Lock is disconnected, and which requires no extra movement of parts in order to further seal the catheter. It is also desirable to have a valve which is inexpensive to manufacture and relatively simple to operate.

### SUMMARY OF THE INVENTION

It is therefore an object of the present invention to provide a device for preventing fluid flow out of a patient when a medical device is withdrawn from a catheter connected to the patient. In one embodiment, the device preferably comprises a tubular catheter hub having an upstream end configured to receive a medical device, and having a catheter cannula attached to a downstream end. The device of the present embodiment also includes an occluder or clamp mounted on the hub and being configured to prevent fluid passage through the cannula. The occluder is preferably biased into an occluding position and requires a positive act to move it into a position in which fluid can flow through the cannula.

In another embodiment, the clamp includes a flexible arm supporting a clamping jaw which is biased into a position to squeeze the cannula to prevent fluid flow therethrough. The arm may be self-biased into said closed position, or a resilient element which biases the arm and jaw into the closed position may be included.

In another embodiment, the device may include a pair of opposing clamping surfaces for pinching the flexible cannula, at least one of the surfaces being a portion of the occluder. The occluder may have a pair of jaws for pinching the flexible cannula, and the jaws may be normally biased into the pinching position. One of the clamping surfaces may be fixed to the hub.

The occluder may be movably mounted on the hub, and the occluder and the hub may be configured so that the connection of a medical device to the upstream end of the hub will move the occluder into an open position wherein it is not restricting flow through the flexible valve portion. The occluder and hub preferably include engaging surfaces that will move the occluder into an open position when the occluder is moved in a downstream direction. The inter-engaging surfaces may cause the flexible valve arm on the occluder to be biased radially outwardly so as to not pinch the cannula. The hub of the device may include guides which guide movement of the occluder into linear movement and restrict rotational movement. The hub upstream end may be configured so that the connection of a medical device to the hub upstream end will automatically cause the occluder to move in a downstream direction relative to the hub and into an open position.

The cannula may include a rigid section extending into the hub and a flexible portion extending further into the hub and in position to be engaged by the occluder. The occluder may include a jaw normally biased into a position to press the flexible section of the cannula into engagement with a surface on the hub. The occluder and the hub may include inter-engaging cams configured to move a jaw on the occluder radially outwardly from the cannula when the occluder is moved in a downstream direction. The occluder is preferably movable between a first position in which it pinches the cannula closed and a second position in which the pinching surfaces are separated sufficiently to allow fluid to flow through the cannula.

Another object of the present invention is to provide a method of controlling fluid flow through a catheter to prevent fluid from flowing out of a patient when a medical device is disconnected from the catheter. In one embodiment, the method comprises the steps of: connecting a medical device to an upstream end of a catheter hub while moving an occluder into an open position in which it does not occlude flow through the catheter, the occluder being normally biased into a closed position wherein it prevents flow through the catheter; and disconnecting the medical device from the catheter allowing the occluder to move into its normally closed position, thereby automatically preventing fluid flow out of a patient when the medical device is withdrawn.

In another embodiment, a method of preventing fluid flow out of a patient when a medical device is withdrawn from a catheter connected to a patient may comprise the steps of: providing a tubular catheter hub having one end adapted to receive a medical device and having a catheter cannula attached to the second end; and providing an occluder slidably mounted on the hub; and normally biasing the occluder into a position in which it pinches the cannula so as to prevent fluid flow through the cannula. The method of the present embodiment further comprises configuring the occluder in the hub so that when a medical device is connected to the first end of the hub, the occluder is moved into an open position in which fluid flow is allowed through the cannula. According to this embodiment, the occluder will automatically move to a closed position when the medical device is withdrawn from the hub.

For purposes of summarizing the invention and the advantages achieved over the prior art, certain objects and advantages of the invention have been described herein above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

All of these embodiments are intended to be within the scope of the present invention herein disclosed. These and other embodiments of the present invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

Having thus summarized the general nature of the invention and its essential features and advantages, certain preferred embodiments and modifications thereof will become apparent to those skilled in the art from the detailed description herein having reference to the figures that follow, of which:

FIG. 1 is a side view of a catheter shut-off valve having features and advantages of the present invention shown in the closed position;

FIG. 2 is a side view of the catheter shut-off valve of FIG. 1 shown in the open position, and having a medical device attached;

FIG. 3 is a top view of the catheter shut-off valve of FIG. 1;

FIG. 4 is a side section view of the catheter shut-off valve taken along line 4-4 of FIG. 3;

FIG. 5 is a catheter hub member of the catheter shut-off valve of FIG. 1;

FIG. 6 is a cross-sectional view of the catheter hub member on line 6-6 of FIG. 5;

FIG. 7 is a cross-sectional view of the catheter hub member on line 7-7 of FIG. 5;

FIG. 8 is an alternative embodiment of the catheter hub member of the catheter shut-off valve of FIG. 1;

FIG. 9 is a cross-sectional view of the catheter hub member on line 9-9 of FIG. 8;

FIG. 10 is a side view of the occluder of the catheter shut-off valve of FIG. 1;

FIG. 11a is a front end view of the occluder of FIG. 10;

FIG. 11b is a front end view of an alternative embodiment of the occluder of FIG. 10;

FIG. 12a is a is a rear end view of the occluder of FIG. 10;

FIG. 12b is a is a rear end view of an alternative embodiment of the occluder of FIG. 10;

FIG. 13 is a side view of an alternative embodiment of a catheter shut-off valve having features and advantages of the present invention shown in the closed position;

FIG. 14 is a top view of the catheter shut-off valve of FIG. 13;

FIG. 15 is a side cutaway view of the catheter shut-off valve of FIG. 13, shown in the closed position;

FIG. 16 is a side cutaway view of the catheter shut-off valve of FIG. 13, shown in an open position;

FIG. 17 is a cross section of another embodiment of the invention employing a threaded coupling for receiving a flange on a catheter connected to a patient; and

FIGS. 18-22 illustrate various alternative forms of resilient elements for urging clamp jaws into a closed position.

### DETAILED DESCRIPTION OF THE PREFERRED EMBODIMENT

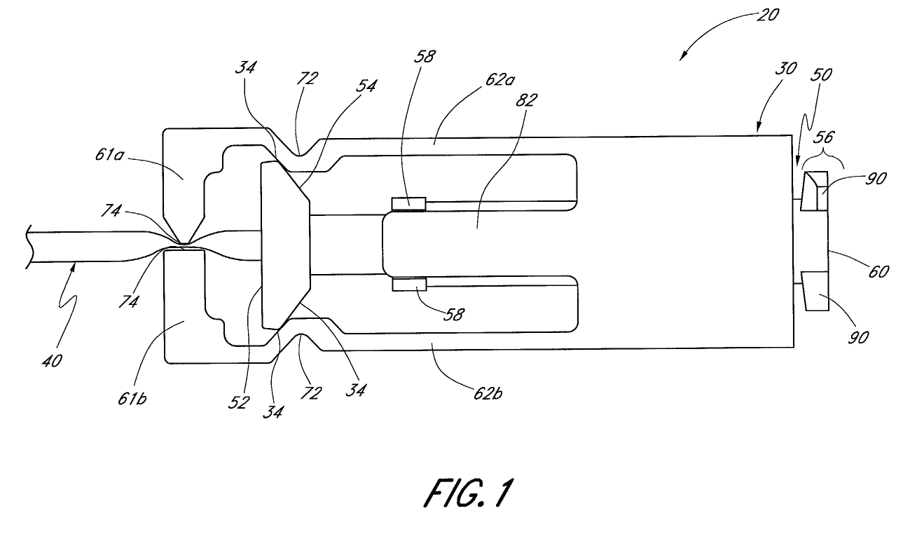

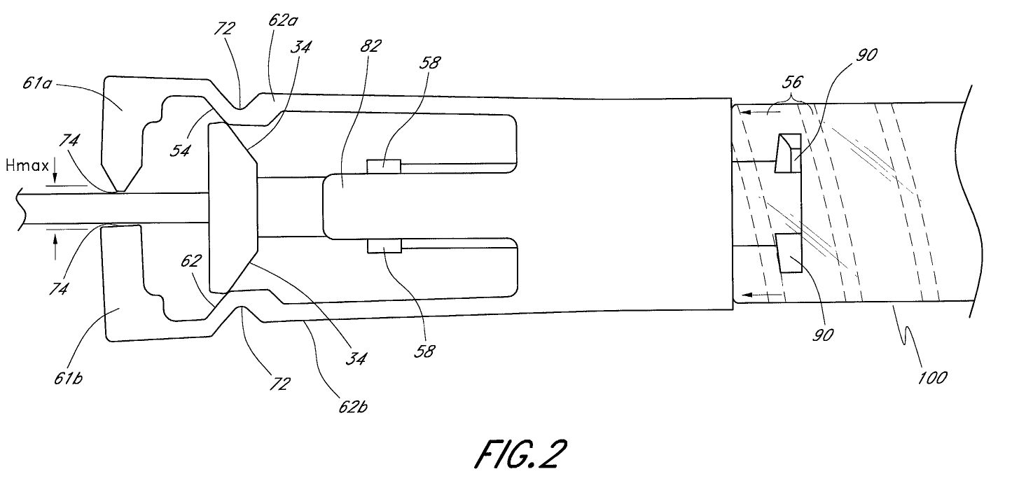

FIGS. 1 and 2 show a catheter shut-off device or valve 20 having features and advantages of the present invention. The valve 20 preferably has two main components; an occluder or clamp 30, and a tubular catheter hub 50. The catheter hub 50 preferably has a flexible, resilient cannula 40 disposed on its end 52, which is downstream with respect to fluid flow into a patient. An interface 56 suitable for receiving a medical device 100 is at the hub rear or upstream end 60. A pair of circumferentially spaced cams 34 extend outwardly from the downstream end of the hub 50. The occluder 30 is disposed such that it is co-axial with the catheter hub 50 and is free to move linearly along the shared axis relative to the catheter hub 50. The occluder 30 preferably has opposing jaws 61a and 61b through which the cannula 40 extends. The jaw members 61a and 61b are biased toward a closed or occluded position in which they “pinch” the soft tubing of the cannula 40, thereby closing the valve.

The outer member or occluder 30 and catheter hub 50 are preferably made of molded polycarbonate or alternatively from any other material suitable for use in medical applications, and capable of providing the features and advantages of the present invention.

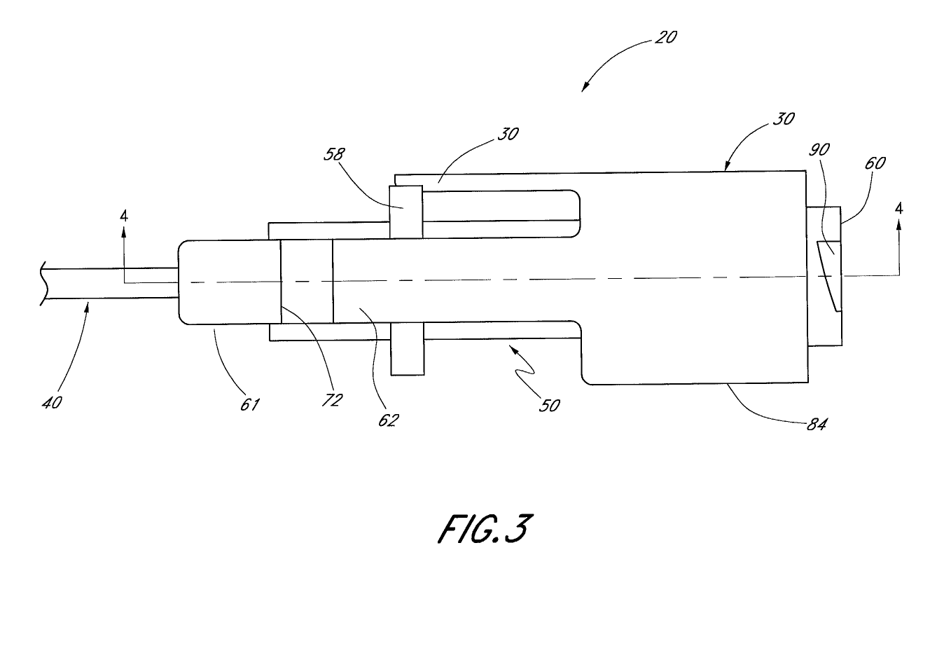

FIGS. 1-4 show the occluder 30 as it is preferably disposed relative to the catheter hub 50. The cannula 40 attached to the catheter hub 50 extends forward through the space between the jaws 61a and 61b, thus placing the occluder 30 and cannula 40 in an operative relationship. The outer member 30 has a pair of cams 54 which mate with the cams 34. The catheter hub 50 and the occluder 30 are disposed relative to one another such that when a medical device 100 is attached to the interface 56 of the hub 50, the occluder 30 is pushed forward relative to the catheter hub 50 such that the cam surfaces 34 and 54 engage, thus causing the jaws 61a and 61b to separate, releasing the cannula 40 and allowing fluid to flow bi-directionally through the catheter 40. This position, with the jaw members 61a and 61b separated (shown in FIG. 2), is referred to herein as the open position. When the medical device 100 is removed from the interface 56 of the hub 50, the bias of the clamping arms 62a and 62b causes the jaw members 61a and 61b to pinch the cannula 40, thus closing the valve 20. This position, shown in FIGS. 1 and 3a, is referred to herein as the closed position.

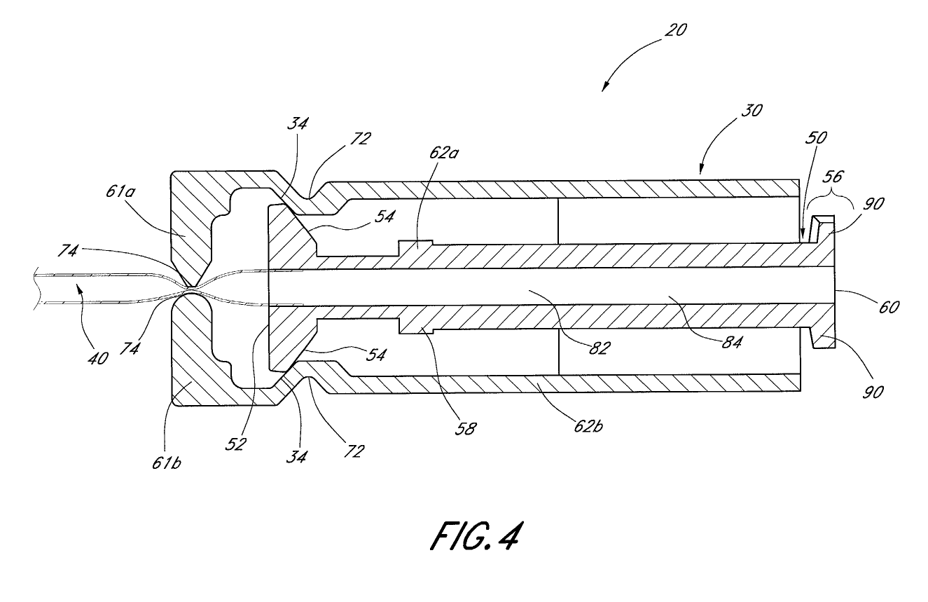

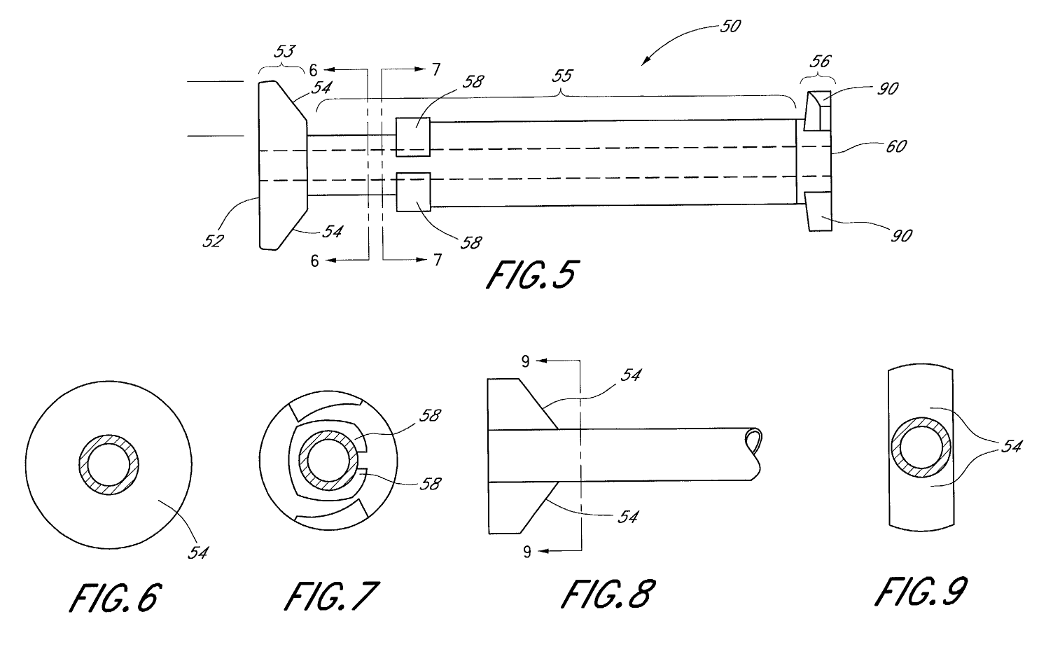

One embodiment of a hub 50 having features and advantages of the present invention is shown in FIGS. 4-7. The hub 50 of this embodiment preferably comprises a substantially tubular member having a cam 54 near its front end 52, a central section 55, and an interface section 56 near its rear end 60. Alternatively, the cam section 54 may be advantageously located at other points along the length of the catheter hub 50 such that features and advantages of the present invention are realized.

As shown in FIG. 4, the hub 50 has an internal passage 82 to provide fluid communication between a medical device (100 FIG. 2) attached at the interface 56 and the cannula attached at the downstream end 52 of the hub 52. The cannula 40 is preferably retained within the hub 50 such that fluid communication is facilitated between the interface section 56 of the hub 50 and the cannula 40. The cannula 40 may be secured to the catheter hub using any suitable method known to those skilled in the art. For example, the cannula 40 may be glued, sonic welded, or frictionally retained on or within the hub 50.

As shown in FIG. 5, the cam section 53 of the catheter hub 50 preferably comprises a cam surface 54 in the form of a section with sides sloping radially and in the downstream direction. The cam surface is preferably annular, as seen in FIG. 6, but need only extend circumferentially sufficiently to engage the mating cam on the occluder 30. The height ‘h’ 92 of the cam section is preferably at least as great as the amount of separation of the jaw members (61a, 61b FIG. 2) required in order to release the cannula 40 (FIG. 2), thereby opening the valve 20. As shown in FIG. 6, the cam surface may extend about the circumference of the catheter hub 50.

Alternatively, as shown in FIGS. 8 & 9, the cam surface 54 may comprise two separate sloped sections 54a and 54b extending radially outward from the surface of the hub 50.

The central section 55 of the catheter hub 50 includes guides 58 which extend outward from the tubular portion of the hub. The guides 58 are disposed such that they interact with the axial guide 82 of the occluder 30 (FIG. 2) in such a way as to restrict rotation of the catheter hub 50 and occluder 30 relative to one another. Additionally, the guides 58 may be used to guide the relative linear motion of the two parts 30 and 50 in order to operate the valve 20 (FIG. 2) in accordance with the present invention.

In the embodiment shown in FIG. 5, the interface section 56 near the rear 60 of the catheter hub 50 is preferably adapted such that a device 100 (FIG. 2) may threadably engage the catheter hub, thus securing the device 100 to the catheter hub 50 and placing the device in fluid communication with the catheter hub 50. The interface 56 preferably comprises a pair of lugs 90 which may threadably engage the medical device 100 such as an ISO standard syringe. The interface 56 may be a Luer lock fitting of the type set forth in American National Standard Institute No. ANSI/HIM MD70.1-983 which is incorporated herein by reference.

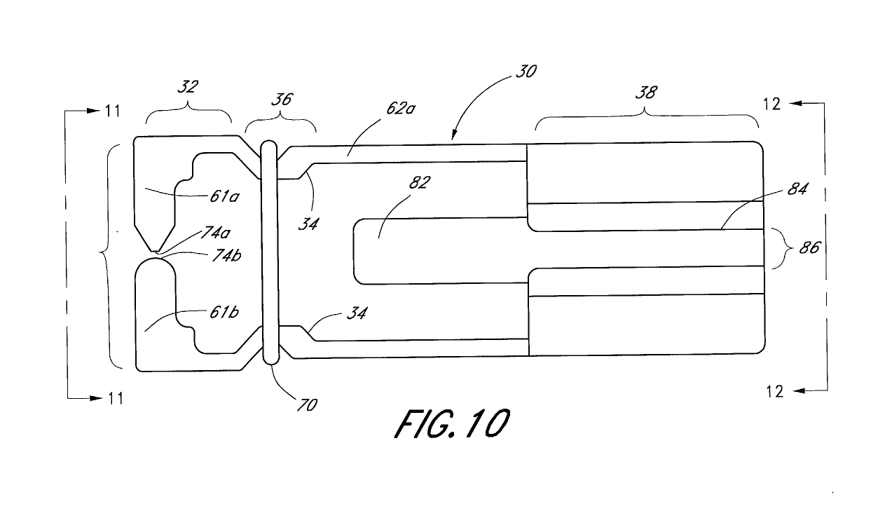

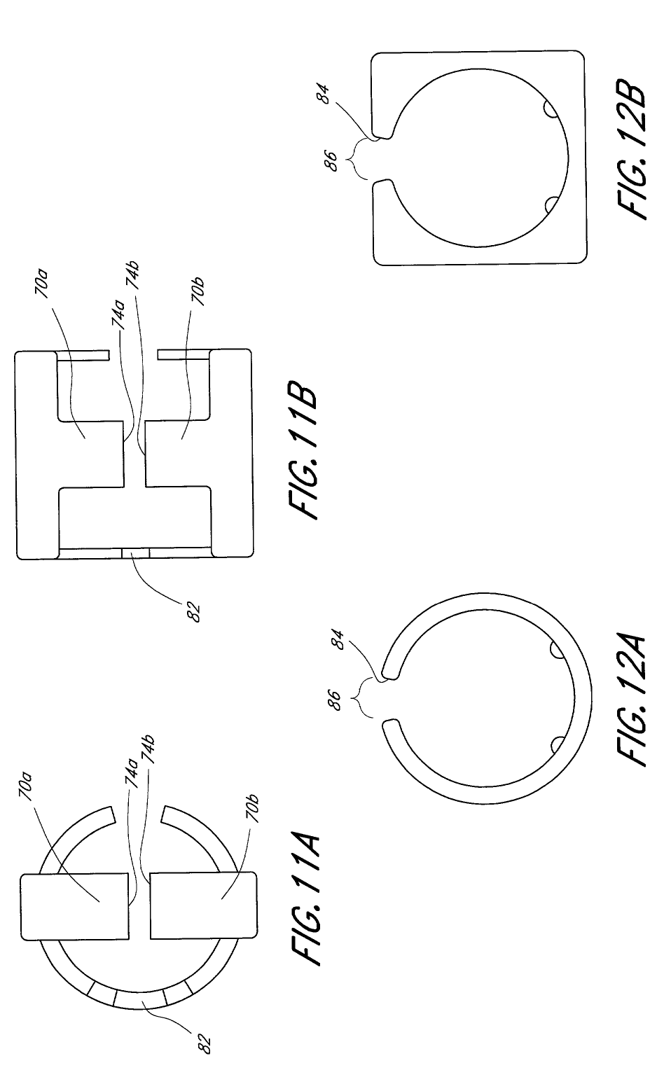

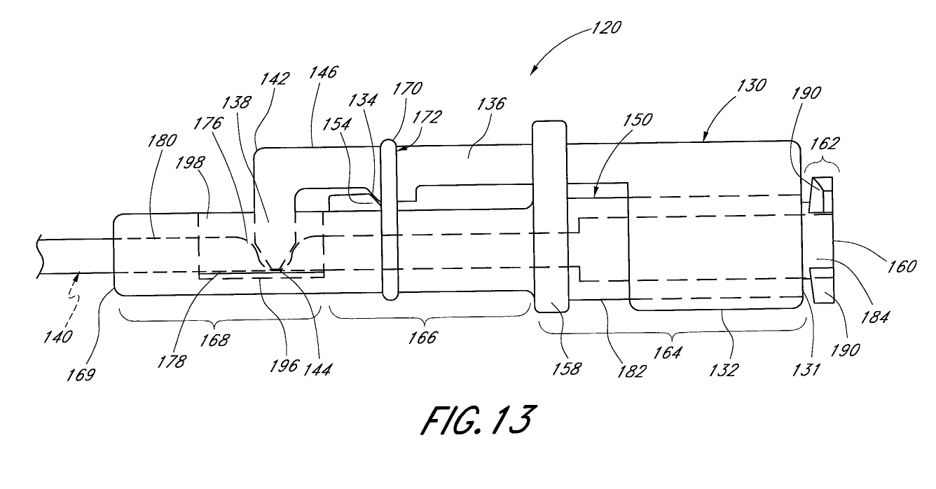

FIGS. 10-12b show the occluder 30 separated from the hub and having features and advantages of the present invention. The occluder 30 has a clamping section 32 near its forwardmost end 31, a cam section 36 rearward therefrom, and an attachment section 38 at the rearmost end of the occluder 30. As shown in FIGS. 11 a and 12a, the occluder 30 preferably comprises a substantially circular cross section with a gap or space 86 formed by edges 84. Alternatively, the occluder 30 may comprise a substantially rectangular cross section as shown in FIGS. 11b and 12b. As will be recognized by those skilled in the art, other cross-sectional shapes may be used without departing from the spirit of the present invention.

The cam section 36 of the occluder 30 is preferably characterized by cam surfaces 34 on the inside of the top and bottom clamping arms 62a and 62b respectively. The cam surfaces 34 are preferably sized and shaped such that they may cause the jaws 61a and 61b to separate when a medical device is attached to the interface 36 section of the catheter hub 50, thus opening the valve 20 as described above with reference to FIGS. 1 and 2.

The clamping section 32 of the present embodiment, as best seen in FIG. 10 preferably comprises a pair of jaws 61a and 61b attached to clamping arms 62a and 62b which are biased towards a closed position. The bias is preferably caused by the resilience of the material. Additionally, an O-ring 70 may be disposed in grooves 72 on the clamping arms 62a and 62b. The O-ring 70 is preferably made of a substantially resilient material such as rubber. The bias created by the clamping arms 62a and 62b, or the combination of the O-ring 70 and clamping arms 62a and 62b, is preferably sufficient to provide a clamping force to pinch closed the soft cannula 40 extending from the front 52 of the catheter hub 50 (FIG. 2).

The clamping edges 74a and 74b of the jaw members 61a and 61b preferably comprise shapes such that they may advantageously close a catheter cannula 40 as described herein. In one preferred embodiment, as best seen in FIG. 4, the clamping edge 74a of the jaw 61a preferably has a substantially convex arch shape. The clamping edge 74b of the jaw 61b has a corresponding concave arch shape when seen in end views (FIGS. 11a and 11b), and a substantially convex shape when seen in side view (FIG. 10). The arch-shaped clamping surfaces 74a and 74b are believed to provide optimal closure of the cannula 40. The clamping surfaces may alternatively be substantially flat (as shown in FIG. 2) or otherwise shaped such that they will pinch the cannula 40 closed as shown and described herein.

As shown in FIG. 10, the occluder 30 preferably comprises an attachment section 38 having the gap 86 sized such that the catheter hub 50 (FIG. 5) may “snap” into the occluder 30 in order to place the two parts 30 and 50 in the position shown in FIG. 1.

As shown in FIG. 10 the occluder 30 includes a guide 82 which extends axially toward the clamping section 32 of the occluder 30. If desired, the guide 82 may be sized and disposed such that it may interact with guide 58 on the catheter hub 50 (FIG. 1) in order to restrain the catheter hub 50 and the occluder 30 from rotation relative to one another. As best seen in FIG. 6b, the end of the attachment section 38 preferably comprises a substantially circular opening into which the catheter hub 50 is positioned after assembly.

FIGS. 13-17 show an alternative embodiment of a catheter shut-off valve 120 having features and advantages of the present invention. This embodiment generally comprises an occluder 130 and a hub 150 preferably disposed such that they may be moved linearly relative to one another in order to allow interactions causing the valve 120 to be opened and closed as described below. The occluder 130, in the closed position in FIG. 13, includes a jaw 138 and pinches a section of soft tubing 176 disposed therein against an internal surface of the hub 150. When a medical device 100 (FIG. 16) is attached to the hub member 150, the occluder 130 is pushed forward relative to the hub 150, causing the section of tubing 176 to be released, thereby opening the valve 120.

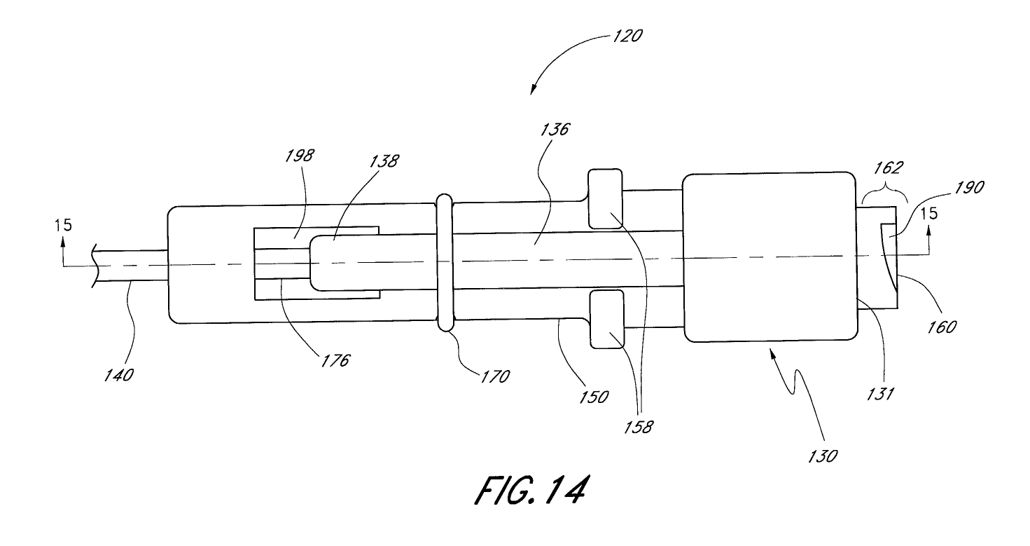

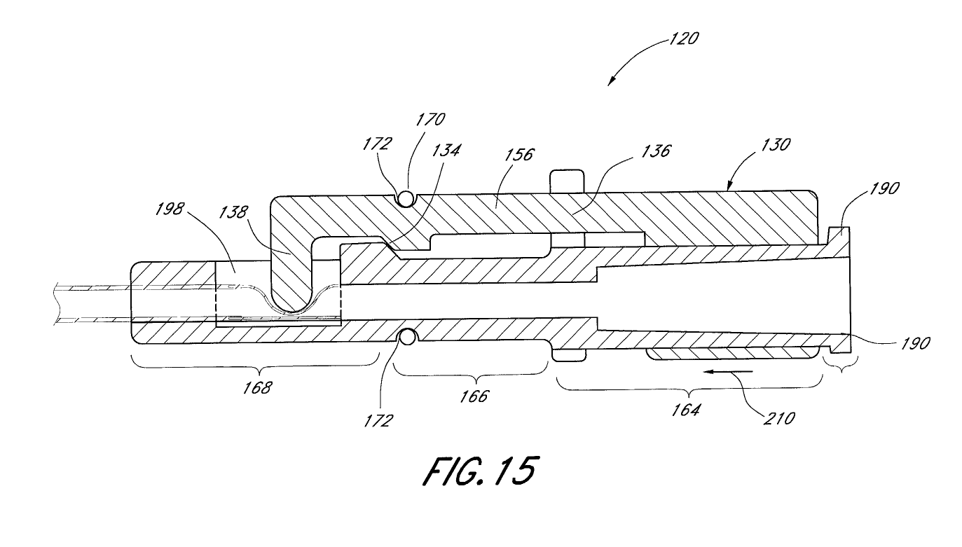

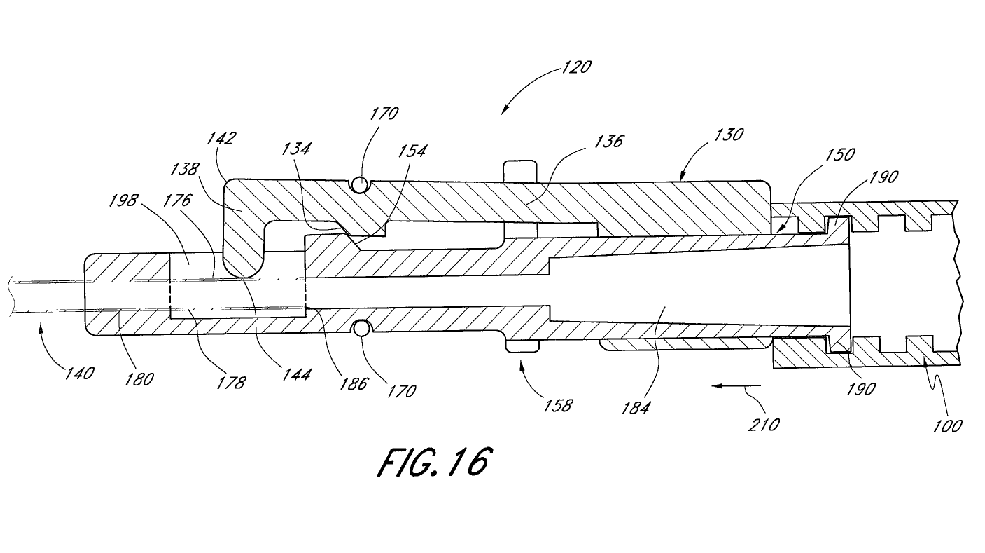

Referring to FIGS. 13 and 14, the occluder 130 is preferably characterized by a ring section 132 at its rearmost end 131 and an elongate flexible arm 136 extending forward from the ring section 132. The ring section 132 preferably comprises a substantially circular cross-sectional shape that is open at its lower portion to enable it to be clipped on the hub 13. However, it may comprise any cross sectional shape such that it may interact with the hub member 150 as described herein. The arm 136 which extends forward from the ring section 164 has a cam surface 134 near its downstream end 142. At that end, the arm 136 extends inward towards the hub 150 forming the jaw 138 terminating in a clamping surface 144. The clamping arm preferably comprises a groove 172 formed in the outside surface 146 of the arm 136 near the cam surface 134. The groove 172 is adapted to receive and retain a portion of an O-ring 170 which urges the arm towards the hub 150.

The clamping surface 144 of the jaw 138 preferably has a substantially convex arch-shape when viewed from the side (as seen best in FIG. 14). Alternatively, the clamping surface 144 may comprise a more “pointed” shape as shown in FIG. 13. The clamping surface 144 may alternatively comprise a substantially flat, concave, or other shape such that functions as described herein.

Referring to FIGS. 13-15, a hub 150 having features and advantages of the present invention preferably has an interface section 162 at its rear end 160, a substantially cylindrical rear section 164, a central section 166 of slightly smaller diameter and having a cam surface 154, and a front section 168. A catheter cannula 140 extends downstream from the front 169 of the hub 150. The catheter cannula 140 may be either a substantially rigid tube, a substantially flexible tube, or any other cannula known to those skilled in the art to be suitable for use in medical transmission of fluids. The catheter cannula 140 is bonded within the internal space 180 of the hub 150. A section of substantially flexible tubing 176 is disposed within the hub 150 and joins the catheter cannula 140 at a junction 178.

At the rear of the hub 160, the internal space 180 defines an inlet space 184. The upstream portion of the tubing 176 is bonded to the internal passage of the hub 150. Alternatively, the catheter cannula 140 may comprise a soft tube which extends through the hub 150.

The hub interface section 162 includes lugs 190 which allow a medical device such as a syringe to be spreadably attached thereto. The rear section 164 has an internal lumen 184 open to the passage 160 of the interface section. At the junction between the rear section 164 and the central section 166, there is a guide 158 on the outer surface of the hub and positioned to restrain relative rotation between the two members 130 and 150 about their shared longitudinal axis.

The central section 166 of the hub 150 has a portion of a groove 172 in the outer surface 182 that together with the groove 172 in the arm 136 receives the O-ring 170. The clamp central section 166 has a cam surface 154 which extends out from the outer surface of the hub 150. The cam surface 154 is preferably sized and adapted such that it may interact with a similar cam surface 134 on the occluder 130.

As best seen in FIGS. 14 and 15, the hub has a hole 198 in its top surface near the forward end 169 sized such that the jaw section 138 of the occluder 130 may extend through the hole 198 and compress the exposed portion of tubing 176. An interior surface 196 is preferably substantially flat, and effectively acts as a clamping surface against which the section of tubing 176 will be pinched by the clamping surface 144 of jaw 138 when the valve 120 is in its closed position. Alternatively, the bottom 196 of the hole 198 may comprise a convex or concave arch-shape to provide a substantial seal when pinching the tubing 176.

FIGS. 15-17 illustrate the operation of the valve 120 of the present embodiment. With the medical device 100 removed from the interface portion 162 of the hub member 150 (FIG. 15), the valve 120 is in a closed position. While in the closed position, the bias of the clamping arm 136 and O-ring 170 (if present) causes the clamping surface 144 of the jaw section 138 to press against on the exposed soft tubing section 176 such that fluid flow is fully occluded. As a medical device 100 is attached, as shown in FIG. 16, the occluder 130 is forced forwards in the direction of the arrow 210, and causing the clamping arm jaw 138 to be flexed outwards by the engaging cam surfaces 134 and 154. As the jaw 138 moves outwards, the pressure of the clamping surface 144 on the soft tubing section 176 is released, allowing fluid communication between the medical device 100 and the cannula 140. This second position, shown in FIG. 16 is referred to as the open position.

Upon removal of the medical device 100 from the interface section 162, the bias of the clamp arm 136 and the O-ring 170 moves the arm once more toward the closed position and causes the cam section 134 to slide inwardly on the cam surface 154. The horizontal component of this force causes the occluder 130 to be moved rearward in the opposite direction of the arrow 210 relative to the hub member 150. The guide 158 cooperates with the arm 136 to guide movement of the clamp 130. As the medical device 100 is removed, the occluder 130 returns to its closed position (FIG. 14) with the clamping surface 144 again pinching the soft tubing section 176, thus stopping fluid flow through the hub 150.

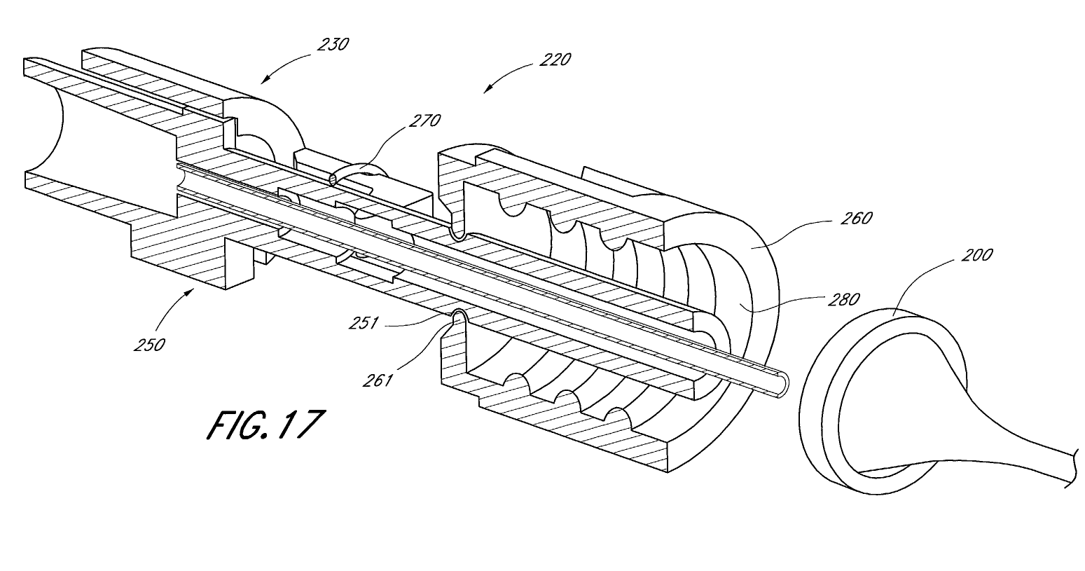

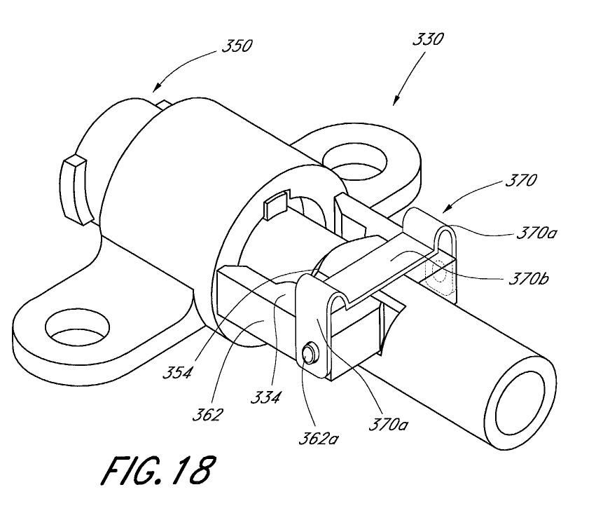

Referring to FIG. 17, another form of the shutoff device or valve 220 of the invention is illustrated. It employs the basic components of the previously described arrangements, including an interior tubular hub 250 and an outer clamp or occluder 230. These components are mounted for slideable relative axial movement to the closed and open positions by a resilient component 270 and interengaging cams. The tubular hub, however, differs from the foregoing arrangements by including an elongated downstream portion on which is rotatably mounted an internally threaded coupling 260. As may be seen, the downstream portion of the hub 250 has an annular groove 251 in its outer surface spaced rearwardly from the downstream end of the hub. The coupling 260 has on its upstream end an inwardly extending annular rib 261 which fits within the groove 251. The threaded interior of the coupling 260 is spaced outwardly from the exterior of the hub, thus creating an annular space 280 for receiving the end of a catheter, schematically indicated at 200. That is, the catheter hub can be inserted into the space 280 and the coupling rotated so as to draw the catheter hub into tight engagement with the hub 270. The catheter interior is of course in communication with the tubing positioned within the clamping device 220.

FIGS. 18-22 illustrate arrangements with alternate elements for urging the jaws closed. FIG. 18 illustrates a resilient element 370 that holds clamp arms 362 in the closed position. An outwardly extending projection or button 362a is formed on each of the clamp arms 362. Opposite ends 370a of the resilient element 370 are snapped on to the buttons 362a while a central section 370b extends between the ends 370a. The resilient element 370 is configured so that the element provides a continual biasing force on the clamp arms 362 into the closed position. Also shown are the interengaging cams 334 and 354 that cause the clamp arm 362 to be forced radially outwardly when a medical device is connected to the upstream end of the hub 350.

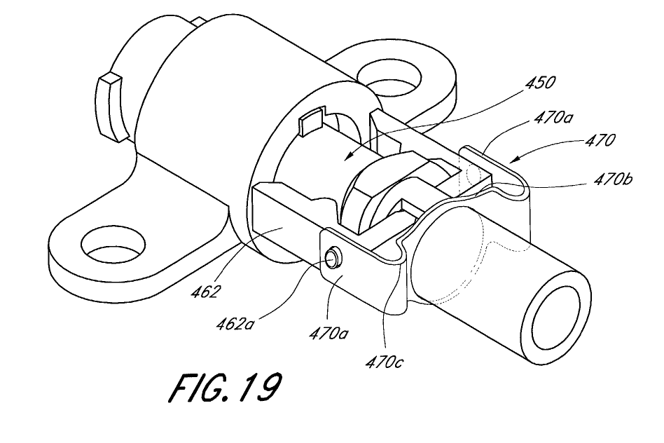

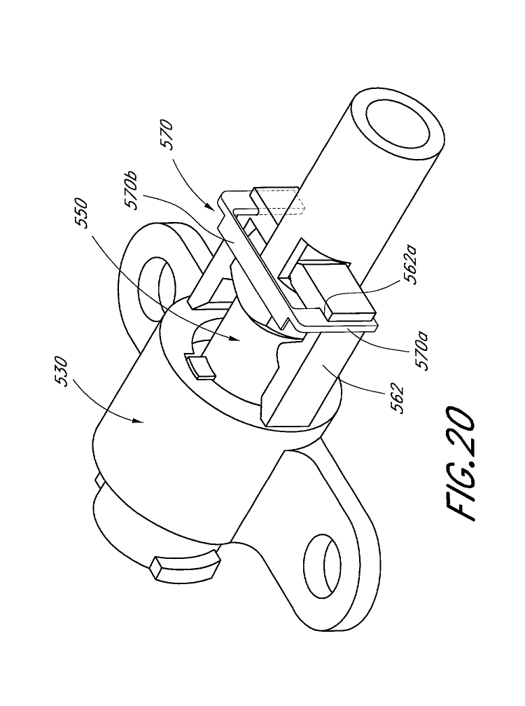

FIG. 19 illustrates a different form of resilient element 470 having generally flat end portions 470a that snap onto buttons 462a formed on the clamp arms 462. These end portions extend generally in an axial direction and include generally U-shaped forward portions 470c that are joined by a central section 470b that is curved to conform to and surround a portion of the hub 450. The U-shaped portions 470c in effect form springs that urge the ends 470a to bias the clamp arms 462 into the closed position.

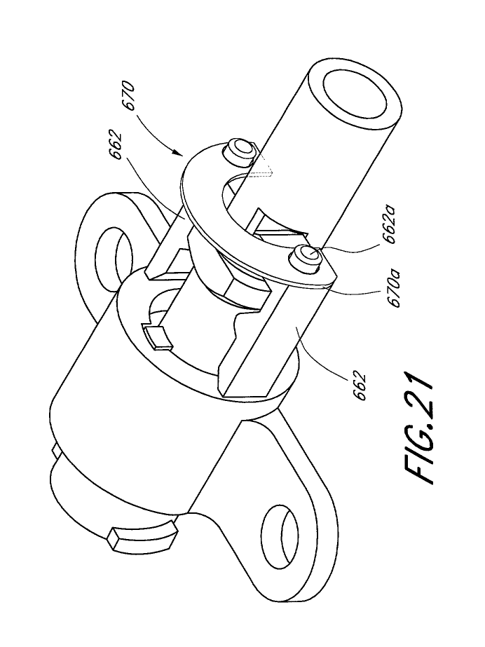

FIG. 20 illustrates yet another embodiment for urging clamp arms 562 into closed position. As can be seen, the outer surface of the downstream end of each clamp arm 562 is formed with an outwardly opening slot 562a. A biasing element 570 straddles the hub 550 and the clamp 530. The element 570 includes generally flat ends 570a, each of which fits into a respective one of the slots 562a in the clamp arms 562. The ends are joined by a flat central section 570b. The biasing element 570 is configured so that the clamp arms 562 are held in the closed position, but can flex to permit the clamp arms to be moved to the open position as relative axial movement of the hub and clamp are caused by the interengaging cams, as previously described.

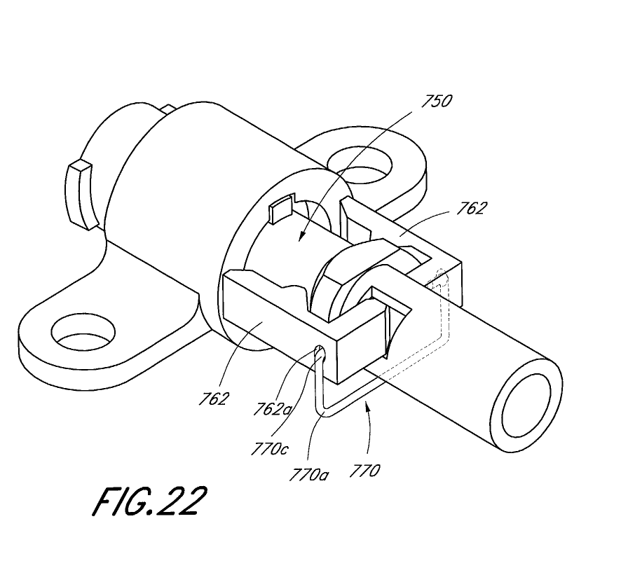

FIG. 21 illustrates yet another form of biasing element 670 which has a generally flat partial disk-shape that has ends 670a, each of which snaps on to a button 662a formed on the downstream end of each clamp arm 662. The element 670 is biased to hold the arms into a closed position but is sufficiently flexible to permit the arms to be moved outwardly.

FIG. 22 illustrates yet another arrangement for biasing clamp arms 762 into a closed position. As can be seen, an element 770 has a rod-like cross section formed into a wide U-shape which straddles the hub 750 and the clamp arms 762. The outer surface of the downstream end of the clamp arm 762 is formed with a socket or hole 762a, and each end 770a of the biasing element 770 is formed with an inwardly extending projection 770c which fits within the socket or hole 762a so as to retain the biasing element 770 on the clamp arms. The biasing element 770 is configured such that it normally holds the clamp arm in a closed position, but will flex to allow the clamp arms to be moved radially outwardly into open position.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described above, but should be determined only by a fair reading of the claims that follow.

What is claimed is:

1. A device for preventing fluid flow out of a patient when a medical component is withdrawn from a catheter connected to the patient, said device comprising:
a tubular hub having an upstream end configured to receive a medical component, and having a catheter cannula attached to a downstream end; and
an occluder mounted on said hub and having a closed position in which it prevents fluid flow through the cannula and an open position in which fluid can flow through the cannula, said occluder being biased into the closed position and requiring a positive force to move it into an open position.

2. The device of claim 1, wherein the occluder includes a clamping jaw which is biased into a position to squeeze the cannula to prevent fluid flow therethrough.

3. The device of claim 2, wherein the occluder includes a flexible arm with said jaw mounted thereon.

4. The device of claim 3, wherein said arm is self-biased into said closed position.

5. The device of claim 2, including a resilient element which biases the jaw into the closed position.

6. The device of claim 5, wherein said resilient element is in the form of an O-ring.

7. The device of claim 2, including means for biasing the jaw into a position to squeeze the cannula to prevent fluid flow therethrough.

8. The device of claim 7, wherein said means is an O-ring.

9. The device of claim 7, wherein said means is in the form of a flexible element that straddles the hub and has one end secured to said jaw, and an opposite end secured to said device in a manner to urge the jaw into position to prevent fluid flow through the cannula, but will flex to permit the jaws to be forced into a position where fluid flow is permitted through the cannula.

10. The device of claim 1, including a pair of opposing clamping surfaces for pinching the cannula, at least one of said surfaces being a portion of said occluder.

11. The device of claim 10, wherein the other one of said surfaces is fixed to said hub.

12. The device of claim 1, wherein the occluder has a pair of jaws for pinching the cannula closed.

13. The device of claim 1, wherein said occluder is movably mounted on said hub, and said occluder and said hub are configured so that the connection of the medical component to the upstream end of the hub will move the occluder into the open position.

14. The device of claim 13, wherein said occluder and said hub include engaging cam surfaces that will move the occluder into an open position when the occluder is moved in a downstream direction relative to the hub.

15. The device of claim 1, wherein said engaging surfaces cause a flexible arm on the occluder to be biased radially outwardly away from the cannula.

16. The device of claim 1, wherein the hub upstream end is configured so that the connection of the device to the hub upstream end will automatically cause the occluder to move in a downstream direction and into the open position.

17. The device of claim 1, wherein said hub includes guides which guide movement of said occluder into linear movement and restrict rotational movement.

18. The device of claim 1, wherein said cannula includes a rigid section extending into said hub and a flexible section extending further into said hub in position to be engaged by said occluder.

19. The device of claim 18, wherein said occluder includes a jaw normally biased into a position to press the flexible section of the cannula into engagement with a surface on said hub.

20. The device of claim 1, wherein said occluder and said hub include cams which are configured to move a jaw on the occluder radially outwardly from the cannula when the occluder is moved in a downstream direction relative to the hub.

21. The device of claim 1, wherein said occluder is axially movable between said closed position in which it pinches the cannula closed and the open position in which the pinching surfaces are separated sufficiently to allow flow through the cannula.

22. A valve to be connected between a catheter and a medical device to prevent fluid from leaking out of the cannula when the device is withdrawn and is disconnected from the catheter, said valve comprising:
a fluid flow passage including a tubular end; and
a valve member for closing the passage;
said end and said valve member being configured so that connecting a medical device to said end causes said valve member to close the passage.

23. A method of controlling fluid flow through a catheter to prevent fluid from flowing out of a patient when a medical component is disconnected from the catheter, said method comprising the steps of:
connecting the medical component to an upstream end of a catheter hub while moving an occluder into an open position in which it does not prevent flow through the catheter, said occluder being normally biased into a closed position wherein it prevents flow through the catheter; and
disconnecting the medical device from the catheter allowing the occluder to move into its normally closed position, thereby automatically preventing fluid flow out of a patient when the medical device is withdrawn.

24. A method of preventing fluid flow out of a patient when a medical component is withdrawn from a catheter connected to a patient, said method comprising the steps of:
providing a tubular hub having one end adapted to receive a medical component and having a second end attached to a catheter cannula; and
providing an occluder slidably mounted on the hub; and
biasing said occluder into a closed position in which it prevents fluid flow through the cannula; and
configuring said occluder and said hub so that when a medical component is connected to said one end of said hub, the occluder is moved into an open position in which fluid flow is allowed through the cannula, and the occluder will automatically move to a closed position preventing fluid flow when the medical device is withdrawn from the hub.

25. A device for preventing fluid flow out of a patient when a medical component is withdrawn from a catheter connected to the patient, said device comprising:
a tubular hub having an upstream end configured to receive a medical component, and having a catheter cannula attached to a downstream end;
a clamp slidably mounted on the hub and including a flexible clamp arm having a clamp jaw thereon for restricting flow through the cannula; and
a biasing element engaging said arm and being configured to bias the arm into position wherein said jaw prevents flow through the cannula.

26. The device of claim 25, wherein said biasing element straddles the hub and the clamp and has an end secured to said arm that urges said jaw into a closed position.
